(19) United States
(12) Patent Application Publication
(10) Pub. No.: US 2024/0050696 A1
(43) Pub. Date: Feb. 15, 2024

(54) CATHETER WITH RAPID EXCHANGE GUIDEWIRE PORT AND METHOD OF MANUFACTURE

(71) Applicant: BOSTON SCIENTIFIC SCIMED, INC., Maple Grove, MN (US)

(72) Inventors: Rachel N. Japuntich, Shoreview, MN (US); Josh Anderson, Coon Rapids, MN (US); Joe Schumacher, Rogers, MN (US); Robert Stemler, Excelsior, MN (US)

(73) Assignee: BOSTON SCIENTIFIC SCIMED, INC., MAPLE GROVE, MN (US)

(21) Appl. No.: 18/232,084

(22) Filed: Aug. 9, 2023

Related U.S. Application Data
(60) Provisional application No. 63/397,526, filed on Aug. 12, 2022.

(51) Int. Cl. A61M 25/00; A61M 25/01

(52) U.S. Cl. CPC A61M 25/0052; A61M 25/01; A61M 2025/0183

(57) ABSTRACT

A rapid exchange catheter and an associated method of forming a port joint for a rapid exchange catheter are disclosed. The catheter includes an elongate shaft, a catheter hub disposed at the proximal end of the elongate shaft, and a guidewire port formed within the elongate shaft at a position distal of the proximal end of the elongate shaft and proximal of the distal end of the elongate shaft. A core wire extends past the guidewire port and includes a distal end region secured to the elongate shaft distal of the guidewire port by virtue of one or more components of the elongate shaft being reflowed around the core wire during manufacture of the rapid exchange catheter.

### CROSS-REFERENCE TO RELATED APPLICATIONS

This application claims the benefit of U.S. Provisional Patent Application Ser. No. 63/397,526, filed Aug. 12, 2022, which is incorporated herein by reference.

### TECHNICAL FIELD

The disclosure relates generally to intracorporeal medical devices and more particularly to intracorporeal medical devices such as rapid exchange catheters, and methods of manufacturing rapid exchange catheters.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a rapid exchange catheter. The rapid exchange catheter includes an elongate shaft extending from a proximal end to a distal end, a catheter hub disposed at the proximal end of the elongate shaft, and a guidewire port formed within the elongate shaft at a position distal of the proximal end of the elongate shaft and proximal of the distal end of the elongate shaft. A core wire extends past the guidewire port from a position proximal of the guidewire port to a position distal of the guidewire port, and the core wire includes a distal end region secured to the elongate shaft distal of the guidewire port.

Alternatively or additionally, the distal end region of the core wire may be secured to the elongate shaft distal of the guidewire port without using any adhesive.

Alternatively or additionally, the distal end region of the core wire may be secured to the elongate shaft distal of the guidewire port by virtue of one or more components of the elongate shaft being reflowed around the core wire during manufacture of the rapid exchange catheter.

Alternatively or additionally, the elongate shaft may include a distal inner shaft having an outer diameter (OD), a distal outer shaft having an inner diameter (ID) and a proximal shaft positioned prior to reflowing with the distal inner shaft disposed within the distal outer shaft and extending proximally from the distal outer shaft, the proximal shaft surrounding a proximal end of the distal outer shaft and adjacent to a proximal end of the distal inner shaft, and the core wire disposed between the OD of the distal inner shaft and the ID of the distal outer shaft.

Alternatively or additionally, wherein prior to reflowing, a port mandrel is inserted through the distal inner shaft, and a crescent-shaped mandrel is inserted between the OD of the distal inner shaft and the ID of the distal outer shaft.

Alternatively or additionally, the elongate shaft may include a distal outer shaft having a lumen, a distal inner shaft extending through the lumen of the distal outer shaft, and a proximal shaft extending proximal of the distal outer shaft and the distal inner shaft, wherein the core wire extends through a lumen of the proximal shaft such that the distal end region of the core wire is secured directly between an outer surface of the distal inner shaft and an inner surface of the distal outer shaft.

Alternatively or additionally, the distal end region of the core wire may be embedded between the distal inner shaft and the distal outer shaft.

Alternatively or additionally, the elongate shaft may include a crescent-shaped lumen extending both proximal and distal of the guidewire port.

Alternatively or additionally, a centroid of the distal end region of the core wire is laterally offset from a longitudinally extending plane passing through both a centroid of the lumen of the distal inner shaft and a centroid of a crescent-shaped lumen formed by the crescent-shaped mandrel.

Alternatively or additionally, a centroid of the distal end region of the core wire is laterally offset from a longitudinally extending plane passing through both a centroid of the elongate shaft and a centroid of the crescent-shaped lumen.

Alternatively or additionally, the crescent-shaped lumen may define a portion of an inflation lumen extending through the elongate shaft to a balloon secured to a distal end of the elongate shaft.

Another example may be found in a rapid exchange catheter. The rapid exchange catheter includes an elongate shaft extending from a proximal end to a distal end and a catheter hub disposed at the proximal end of the elongate shaft. A port joint is formed along the elongate shaft between the proximal end of the elongate shaft and the distal end of the elongate shaft. The elongate shaft includes a proximal shaft extending proximal of the port joint, a distal inner shaft extending distal of the port joint, and a distal outer shaft extending distal of the port joint, wherein a distal end of the proximal shaft is secured to a proximal end of the distal inner shaft and a proximal end of the distal outer shaft at the port joint. A lumen of the distal inner shaft opens to an exterior of the elongate shaft at a guidewire port formed at the port joint and a core wire extends distally past the guidewire port, the core wire including a distal end region secured to the elongate shaft at the port joint.

Alternatively or additionally, the distal end region of the core wire may be secured to the elongate shaft at the port joint without using any adhesive.

Alternatively or additionally, the distal end region of the core wire may be secured to the elongate shaft at the port joint by virtue of a proximal end region of the inner distal shaft and a proximal end region of the outer distal shaft being reflowed around the core wire during manufacture of the port joint.

Alternatively or additionally, in a cross-section taken through the port joint perpendicular to a central longitudinal axis of the elongate shaft, the cross-section may include a guidewire lumen having a centroid spaced away from the central longitudinal axis of the elongate shaft in a first direction from the central longitudinal axis of the elongate shaft and a crescent-shaped inflation lumen having a centroid spaced away from the central longitudinal axis of the elongate shaft in a second direction from the central longitudinal axis of the elongate shaft, the second direction being opposite the first direction.

Alternatively or additionally, in the cross-section, the core wire may have a centroid spaced away from the central longitudinal axis of the elongate shaft in a third direction from the central longitudinal axis of the elongate shaft, the third direction being different from the first direction and the second direction.

Alternatively or additionally, the third direction may be at an acute angle to the first direction.

Another example may be found in a method of manufacturing a catheter having a rapid exchange guidewire port. The method includes forming an assembly by disposing a distal inner shaft having an outer diameter (OD) within a distal outer shaft having an inner diameter (ID), disposing a distal end region of a proximal shaft around a proximal end region of the distal outer shaft with a proximal end region of the distal inner shaft extending adjacent to the distal end region of the proximal shaft, inserting a first mandrel through a lumen of the distal inner shaft, inserting a second mandrel between the OD of the distal inner shaft and the ID of the distal outer shaft, and inserting a core wire between the OD of the distal inner shaft and the ID of the distal outer shaft. Thereafter, heat and pressure are applied to the assembly in order to form the rapid exchange guidewire port.

Alternatively or additionally, the distal end region of the proximal shaft may include an enlarged profile including a tab that extends along the OD of the distal inner shaft when the distal end region of the proximal shaft is disposed around the proximal end region of the distal outer shaft.

Alternatively or additionally, the core wire may extend proximally of the rapid exchange guidewire port.

Alternatively or additionally, applying heat and pressure to the assembly may cause the distal inner shaft, the distal outer shaft and the proximal shaft to melt and reflow around the first mandrel, the second mandrel and the core wire, thereby locking the core wire in place relative to the rapid exchange guidewire port.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a plan view of an illustrative rapid exchange balloon catheter;

FIG. 2 is a cross-sectional view taken along the line 2-2 of FIG. 1;

FIG. 3 is a perspective view of a distal inner shaft extending within a distal outer shaft;

FIG. 4 is a perspective view of the distal inner shaft and distal outer shaft of FIG. 3, with a port mandrel extending within the distal inner shaft and a crescent-shaped mandrel poised exterior thereto;

FIG. 4A is a cross-sectional view taken along the line 4-4 of FIG. 4;

FIG. 5 is a perspective view of the distal inner shaft and distal outer shaft of FIG. 3, with the port mandrel extending within the distal inner shaft, the crescent-shaped mandrel extending within a space between the distal inner shaft and the distal outer shaft, and a proximal shaft poised exterior thereto;

FIG. 5A is a cross-sectional view taken along line 5-5 of FIG. 5;

FIG. 6 is a perspective view of the distal inner shaft and distal outer shaft of FIG. 3, with the port mandrel extending within the distal inner shaft, the crescent-shaped mandrel extending within a space between the distal inner shaft and the distal outer shaft, and the proximal shaft advanced over the distal inner shaft and the distal outer shaft, a portion of the proximal shaft extending within the space between the distal inner shaft and the distal outer shaft, thereby forming an assembly;

FIG. 6A is a cross-sectional view taken along the line 6A-6A of FIG. 6;

FIG. 6B is a cross-sectional view taken along the line 6B-6B of FIG. 6;

FIG. 7 is a perspective view showing a heat shrink tube disposed over the assembly of FIG. 6;

FIG. 7A is a cross-sectional view taken along the line 7-7 of FIG. 7;

FIG. 8 is a perspective view of the assembly and heat shrink tube of FIG. 7, shown after application of heat and pressure; and

FIG. 9 is a perspective view of the assembly and heat shrink tube of FIG. 7, shown after additional application of heat.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

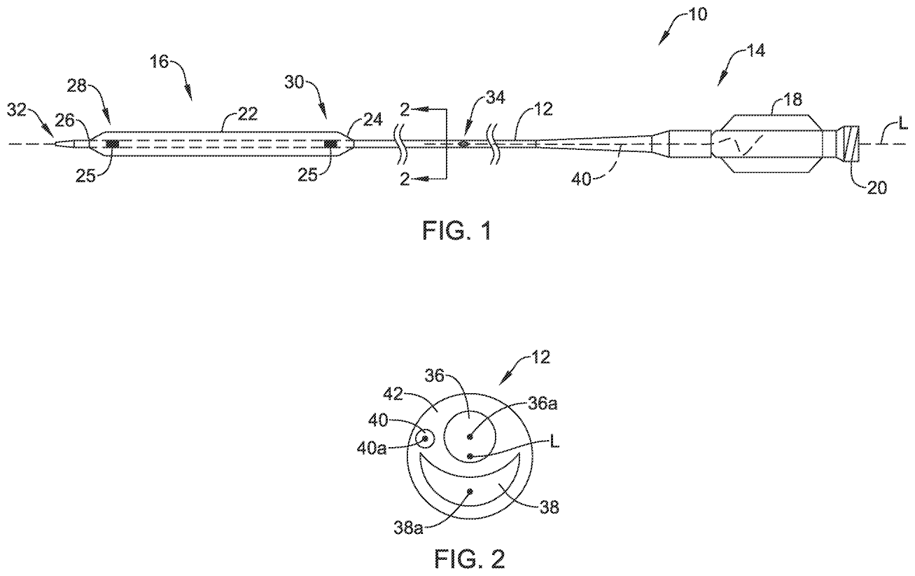

FIG. 1 is a side view of an illustrative catheter 10, for example a balloon catheter. The catheter 10 may include a catheter shaft 12 extending from a proximal region 14 to a distal region 16. A hub 18 may be secured to the catheter shaft 12 at the proximal region 14, with the catheter shaft 12 extending distally from the hub 18. In some cases, the hub 18 may include a Luer fitting 20 that allows other devices to be connected to the hub 18. In some cases, the Luer fitting 20 may be used to fluidly couple the catheter 10 to a source of inflation fluid such as but not limited to saline. The catheter 10 may include an inflatable balloon 22 mounted on the distal region 16 of the catheter shaft 12. The inflatable balloon 22 may be fluidly coupled with an inflation lumen (not shown in FIG. 1) that extends through the catheter shaft 12 and through the hub 18 to the Luer fitting 20. In some cases, the catheter shaft 12 may include an outer tubular member that extends to a proximal waist 24 of the inflatable balloon 22 and an inner tubular member that extends distally beyond the outer tubular member to a distal waist 26 of the inflatable balloon 22. The proximal waist 24 may be secured to the outer surface of the outer tubular member and the distal waist may be secured to the outer surface of the inner tubular member. A variety of construction techniques concerning how the inflatable balloon 22 is made, and how the inflatable balloon 22 is secured relative to the catheter shaft 12 are known, and are applicable here.

In some cases, as shown, one or more marker bands 25 may be secured relative to the catheter shaft 12. In some cases, one of the marker bands 25 may be positioned to fluoroscopically indicate a distal region 28 of the inflatable balloon 22 proximate a distal cone of the inflatable balloon 22 and one of the marker bands 25 may be positioned to fluoroscopically indicate a proximal region 30 of the inflatable balloon 22 proximate a proximal cone of the inflatable balloon 22. This allows a physician or other professional advancing the catheter 10 through a patient's vasculature to an appropriate treatment site to know where the body of the inflatable balloon 22 is located relative to the appropriate treatment site prior to inflating the inflatable balloon 22. In some cases, the catheter 10 may be considered as having a central longitudinal axis L.

In many cases, the catheter 10 may be advanced over a guidewire in order to reach an appropriate treatment site. In some cases, the catheter 10 may have a distal guidewire port 32 located at the distal end of the catheter shaft 12 distal of the inflatable balloon 22 that is adapted to accommodate a guidewire (not shown) extending distally from the distal guidewire port 32. The catheter 10 may have a proximal guidewire port 34 that is proximal of a distal end of the catheter 10 and distal of a proximal end of the catheter 10. The proximal guidewire port 34 may be located a short distance proximal of the inflatable balloon 22 at a location distal of the hub 18. In some instances, given the relative position of the proximal guidewire port 34, the catheter 10 may be considered as being a rapid exchange catheter, meaning that a guidewire only extends through a relatively short portion of the catheter shaft 12 of the catheter 10, instead of extending through an entire length of the catheter shaft 12 of the catheter 10 (which would then be referred to as an OTW, or over-the-wire, catheter. A rapid exchange catheter has benefits including being easier to advance the catheter 10 over the guidewire, withdraw the catheter 10 from the guidewire and exchange the catheter 10 for a different medical device.

FIG. 2 is a cross-sectional view of the shaft 12, taken along the line 2-2 of FIG. 1. It will be appreciated that this cross-section is taken just distal of the proximal guidewire port 34. In some cases, as will be discussed, this cross-section represents several different catheter components fused, bonded or otherwise joined together in a reflow process that forms the proximal guidewire port 34 while maintaining the patency of one or more lumens extending through the catheter shaft 12. A reflow process may involve application of heat and/or pressure in order to cause one or more catheter components or portions of one or more catheter components to melt and reflow, thereby securing the one or more catheter components together when the catheter components cool and solidify. In some cases, the proximal guidewire port 34 itself is formed as part of a reflow process.

As seen in FIG. 2, the catheter shaft 12 includes a guidewire lumen 36. As shown, the guidewire lumen 36 has a circular cross-sectional shape and is sized to receive a guidewire therethrough. The guidewire lumen 36 may be considered as having a centroid 36a that is spaced in a first direction from a point representing the central longitudinal axis L of the catheter shaft 12. The catheter shaft 12 also includes an inflation lumen, which may be a crescent-shaped inflation lumen 38 through the port joint region of the catheter shaft 12 having the proximal guidewire port 34. The crescent-shaped inflation lumen 38 may be considered as having a centroid 38a that is spaced in a second direction from the point representing the central longitudinal axis L of the catheter shaft 12. In some cases, as will be discussed, the guidewire lumen 36 and the crescent-shaped inflation lumen 38 may be formed, and/or the patency of the guidewire lumen 36 and the crescent-shaped inflation lumen 38 may be protected during a reflow process using mandrels that define and/or preserve the guidewire lumen 36 and the crescent-shaped inflation lumen 38 during a reflow process.

The catheter shaft 12 also includes a core wire 40 extending along a length of the catheter shaft 12. The core wire 40 may extend proximally through the catheter shaft 12, and may be secured at its proximal end within the hub 18. The proximal end of the core wire 40 may be adhesively secured within the hub 18. In some cases, the proximal end of the core wire 40 may be secured via a mechanical interference such as but not limited to a frictional fit within the hub 18. A distal end region of the core wire 40 may be secured in place as a result of a reflow process that causes the polymeric material of some of the catheter components to melt and reflow around the distal end region of the core wire 40. The core wire 40 may be considered as having a centroid 40a that is spaced in a third direction from the point representing the central longitudinal axis L of the catheter shaft 12. In some cases, the first direction (spacing of the guidewire lumen 36), the second direction (spacing of the crescent-shaped inflation lumen 38) and the third direction (spacing of the core wire 40) are all different. In some instances, the first direction may be directly opposite the second direction (e.g., 180 degrees apart), while the third direction may extend at an acute angle to the first direction.

The catheter shaft 12, at least in the region identified by the cross-section of FIG. 2, may be considered as a port joint having a catheter body 42 that, as will be discussed, is formed by a reflow process in which one or more catheter components or portions of one or more catheter components are caused to melt and reflow. The catheter body 42, or portions thereof, may therefore be formed from a plurality of different catheter components such as but not limited to shaft portions, that are caused to melt and reflow to join the different catheter components together. Accordingly, the catheter body 42 may be formed of a plurality of different polymer tubes formed of the same or different polymer materials. While the catheter body 42 is shown as a monolithic structure, it will be appreciated that the different polymer materials of the various tubular members forming the catheter body 42 may be uniformly or non-uniformly mixed together. In some cases, the different polymer materials of the various tubular members forming the catheter body 42 may not be uniformly mixed, but instead may be concentrated in particular portions or fractions of the catheter body 42. The following drawings provide an illustrative but non-limiting example of how the catheter body 42, and the proximal guidewire port 34 that is formed within the catheter body 42, may be formed.

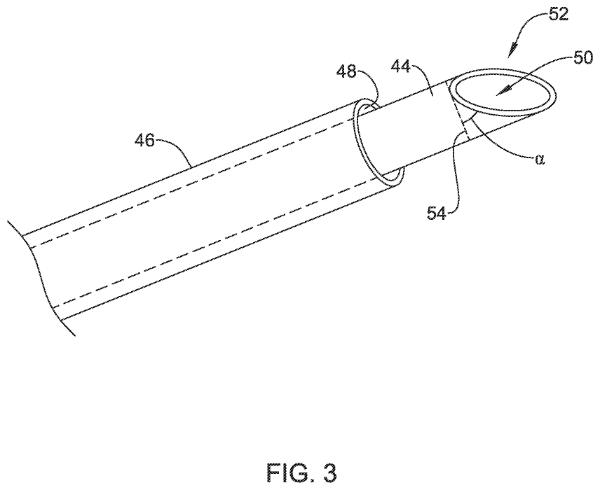

FIG. 3 shows an initial step in forming the catheter body 42 and the proximal guidewire port 34. In FIG. 3, a distal inner tubular shaft 44 may be seen extending within a distal outer tubular shaft 46. In some cases, the distal inner tubular shaft 44 may extend distally through the inflatable balloon 22 and may extend at least to the distal waist 26 of the inflatable balloon 22 to be secured thereto. In some cases, the distal outer tubular shaft 46 may extend distally to the inflatable balloon 22, and may extend at least to the proximal waist 24 of the inflatable balloon 22 to be secured thereto. As an example, the distal waist 26 of the inflatable balloon 22 may be secured to a distal region of the distal inner tubular shaft 44 and the proximal waist 24 of the inflatable balloon 22 may be secured to a distal region of the distal outer tubular shaft 46. The proximal waist 24 of the inflatable balloon 22 may be adhesively secured or heat bonded (e.g., laser bonded or hot jaw bonded) to the distal outer tubular shaft 46, for example. In some cases, the distal waist 26 of the inflatable balloon 22 may be adhesively secured or heat bonded (e.g., laser bonded or hot jaw bonded) to the distal inner tubular shaft 44.

It will be appreciated that the distal inner tubular shaft 44 may have an inner diameter (ID) and an outer diameter (OD). The ID of the distal inner tubular shaft 44 may define a lumen of the distal inner tubular shaft 44. The lumen of the distal inner tubular shaft 44 may be a guidewire lumen extending from the proximal guidewire port 34 to the distal guidewire port 32. The distal outer tubular shaft 46 may have an inner diameter (ID) and an outer diameter (OD). The ID of the distal outer tubular shaft 46 may defined a lumen of the distal outer tubular shaft 44 through which the distal inner tubular shaft 44 extends through. In some cases, a space 48 may be defined between the OD (e.g., the outer surface) of the distal inner shaft 44 and an ID (e.g., the inner surface) of the distal outer shaft 46. The space 48 may be annular, for example, through a portion of the length of the catheter shaft 12 distal of the proximal guidewire port 34 or may have other cross-sectional profiles depending on the position of the distal inner tubular shaft 44 relative to the distal outer tubular shaft 46. The distal inner tubular shaft 44 may move or shift relative to the distal outer tubular shaft 46, depending on whether any mandrels have been inserted yet, for example. The distal inner tubular shaft 44 may have an ID that ranges from about 0.008 inches to about 0.5 inches and an OD that ranges from about 0.009 inches to about 0.6 inches. The distal outer tubular shaft 46 may have an ID that ranges from about 0.01 inches to about 0.61 inches and an OD that ranges from about 0.011 inches to about 0.62 inches. Due to a difference between the OD of the distal inner tubular shaft 44 and the ID of the distal outer tubular shaft 46, the annular space 48 between the OD of the distal inner tubular shaft 44 and the ID of the distal outer tubular shaft may be about 0.001 inches to about 0.6 inches, assuming that the distal inner tubular shaft 44 remains centered within the distal outer tubular shaft 46.

A lumen 50 is defined by and extends through the distal inner tubular shaft 44. The lumen 50 may form a guidewire lumen extending through the distal inner tubular shaft 44 from the proximal guidewire port 34 to the distal guidewire port 32. The distal inner tubular shaft 44 may have a proximal end 52 that is cut at an acute angle α (alpha) with respect to a transverse plane 54 perpendicular to the central longitudinal axis of the distal inner tubular shaft 44. The acute angle α may range from 10 to 80 degrees, for example. In other instances, the proximal end 52 may be cut perpendicular to the central longitudinal axis of the distal inner tubular shaft 44. The proximal end 52 of the distal inner tubular shaft 44 may form part of the proximal guidewire port 34 as discussed further herein. The distal inner tubular shaft 44 may be formed of any suitable polymers, including those listed below. The distal outer tubular shaft 46 may be formed of any suitable polymers, including those listed below.

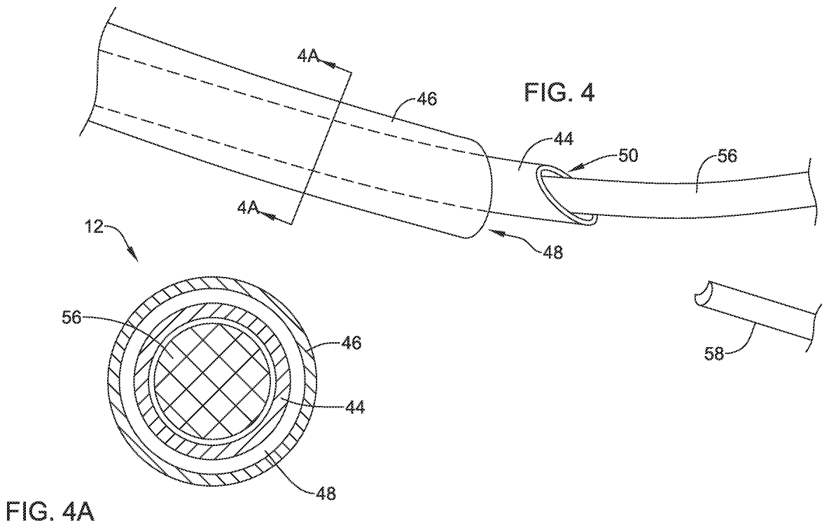

FIG. 4 shows the addition of several mandrels to what is shown in FIG. 3. FIG. 4A is a cross-sectional view taken along the line 4A-4A of FIG. 4. As shown, a guidewire port mandrel 56 has been inserted into the lumen 50 that is defined by the distal inner tubular shaft 44. In some cases, and as can be seen in FIG. 4A, the port mandrel 56 may have a circular cross-sectional profile. As a result, the port mandrel 56 maintains the lumen 50 extending within the distal inner tubular shaft 44 during a subsequent heating of a reflowing process, as described herein. The port mandrel 56 may be formed of any suitable material that can withstand the heat and pressure applied in the subsequent reflow process. As an example, the port mandrel 56 may be formed of a metal such as stainless steel. A crescent-shaped mandrel 58 has not yet been inserted, but is poised for insertion into the space 48 defined between the outer surface of the distal inner tubular shaft 44 and the inner surface of the distal outer tubular shaft 46.

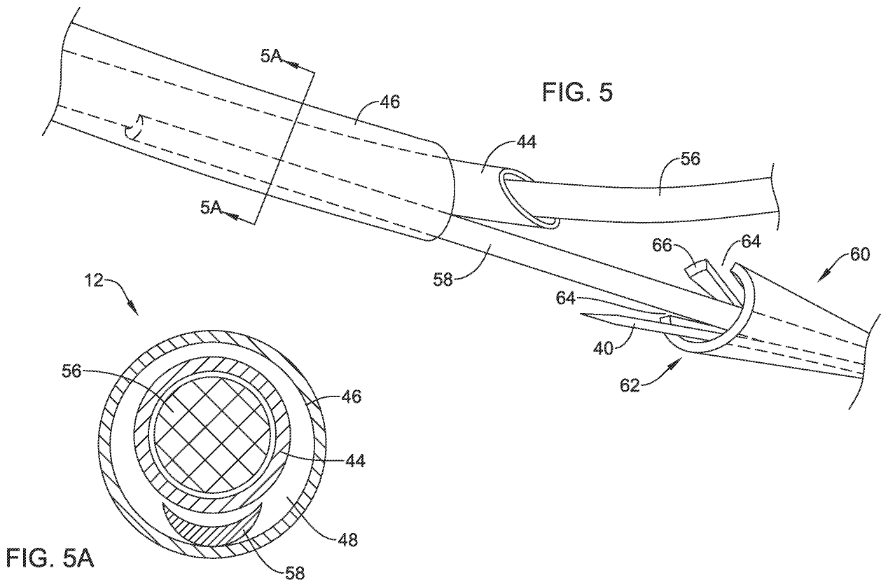

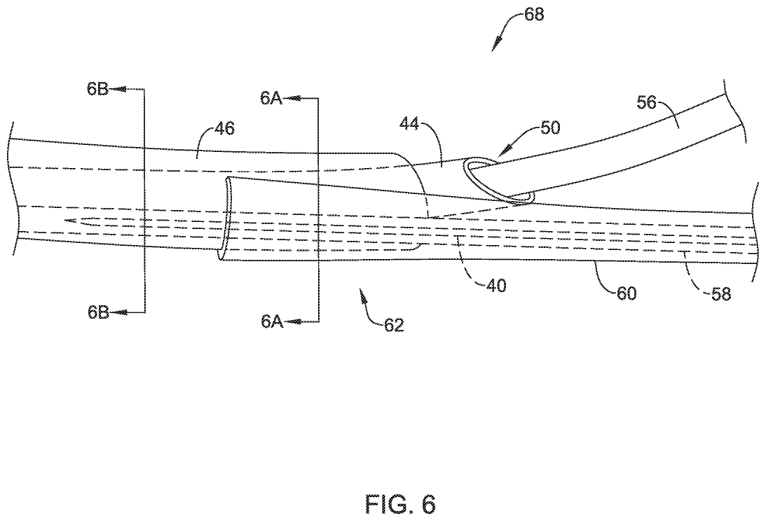

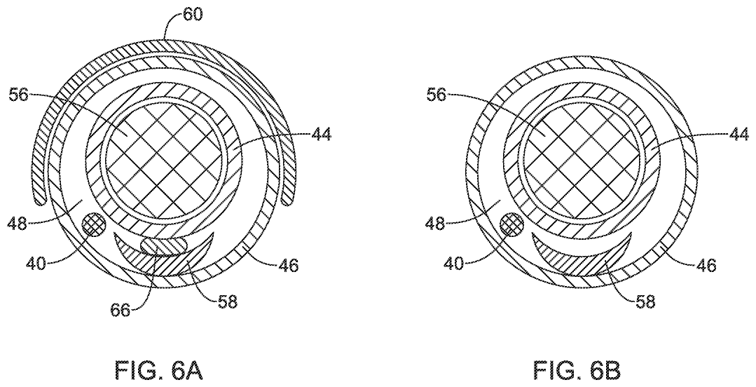

In FIG. 5, the crescent-shaped mandrel 58 has been inserted into the annular space 48 between the outer surface of the distal inner tubular shaft 44 and the inner surface of the distal outer tubular shaft 46. A proximal shaft 60 is poised to be positioned relative to the other components. The lumen of the proximal shaft 60 may define an inflation lumen in fluid communication with the hub 18 for providing an inflation fluid to the interior of the inflatable balloon 22. Upon securing the proximal shaft to the distal inner and outer shafts at the port joint, described further herein, the lumen of the proximal shaft 60 may be in fluid communication with the crescent-shaped inflation lumen 38 extending through the catheter body 42 at the port joint and the inflation lumen defined by the annular space between the outer surface of the distal inner shaft 44 and the inner surface of the distal outer shaft 46 extending distal of the port joint to the inflatable balloon 22. The proximal shaft 60 may have a flared distal end region 62 to facilitate insertion of the distal outer tubular shaft 46 into the distal end region 62 of the proximal shaft 60 such that the distal end region 62 surrounds the proximal end region of the distal outer tubular shaft 46. Accordingly, the flared distal end region 62 may help the proximal shaft 60 to fit around the distal outer shaft 46, for example. In some cases, as shown, the distal end region 62 of the proximal shaft 60 includes a pair of slots 64 that define a tab 66 therebetween that is part of the distal end region 62 of the proximal shaft 60. In some cases, as will be shown with respect to FIG. 6A, the tab 66 may extend into the space 48 between the outer surface of the distal inner tubular shaft 44 and the inner surface of the distal outer tubular shaft 46 with the distal inner tubular shaft 44 extending along an exterior surface of the tab 66 prior to the reflowing process.

In some instances, the core wire 40 may extend through the lumen of the proximal shaft 60 and distal of the distal end of the proximal shaft 60 prior to joining the proximal shaft 60 to the distal inner and outer shafts 44, 46. For example, the hub 18 may be secured to the proximal end of the proximal shaft and the proximal end of the core wire 40 may be secured within the hub 18 with the core wire 40 extending distally through the lumen of the proximal shaft 60 prior to joining the distal inner and outer shafts 44, 46 to the proximal shaft 60.

In FIG. 6, the proximal shaft 60 has been advanced distally over the distal outer tubular shaft 46 with the crescent-shaped mandrel 58 extending through the lumen of the proximal shaft 60 into the space 48 between the outer surface of the distal inner tubular shaft 44 and the inner surface of the distal outer tubular shaft 46, while the proximal end region of the distal inner shaft 44 extends along the exterior of the tab 66 such that the tab 66 is juxtaposed with the crescent shaped mandrel 58 as shown in FIG. 6A. The port mandrel 56 extends proximally from the lumen 50 of the distal inner tubular shaft 44 along an exterior of the distal end region of the proximal tubular shaft 60. As can be seen for example in FIG. 6A, the tab 66 is inserted into the space 48 between the distal inner tubular shaft 44 and the distal outer tubular shaft 46 alongside and facing the crescent-shaped mandrel 58. As shown, the tab 66 is disposed between the convex outer surface of the distal inner shaft 44 and the concave surface of the crescent-shaped mandrel 58. In some cases, the tab 66 may be disposed to a side of the crescent-shaped mandrel 58 (with respect to the illustrated orientation), if desired.

Furthermore, the distal end region of the core wire 40 may be inserted into the space 48 between the distal inner tubular shaft 44 and the distal outer tubular shaft 46 alongside the crescent-shaped mandrel 58 as the distal end region of the proximal shaft 60 is advanced distally around the proximal end region of the distal outer shaft 46. The core wire 40 may be seen disposed within the space 48 between the outer surface of the distal inner tubular shaft 44 and the inner surface of the distal outer tubular shaft 46, shown in FIG. 6A. As noted, the core wire 40 extends through the lumen of the proximal shaft 60 from the hub 18. The exact position of the core wire 40 is merely illustrative, as the core wire 40 could be on an opposing side of the crescent-shaped mandrel 58, for example. FIG. 6 may be considered as showing an assembly 68, which includes the distal inner shaft 44 disposed within the distal outer shaft 46, the port mandrel 56 disposed within the lumen 50 of the distal inner shaft 44, the crescent-shaped mandrel 58 disposed within the space 48 between the distal inner shaft 44 and the distal outer shaft 46, the core wire 40 extending within the proximal shaft 60 into the space 48 between the distal inner shaft 44 and the distal outer shaft 46, the flared distal end region 62 of the proximal shaft 60 extending around the proximal end region of the distal outer shaft 46, and the proximal end region of the distal inner shaft 44 extending along an exterior of the tab 66 of the proximal shaft 60. The assembly 68 may be subjected to heating and/or pressure in order to cause a reflow process.

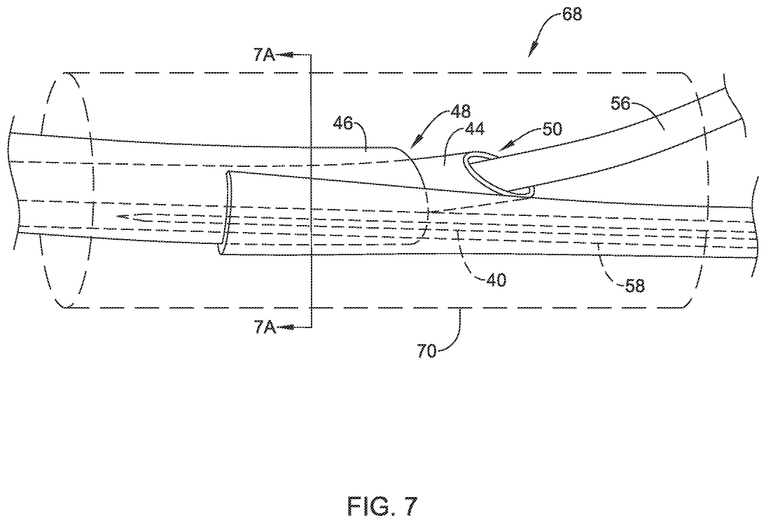

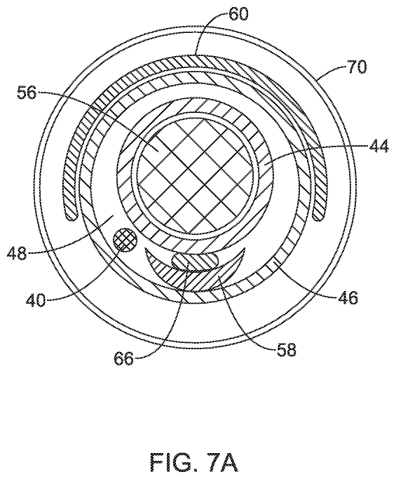

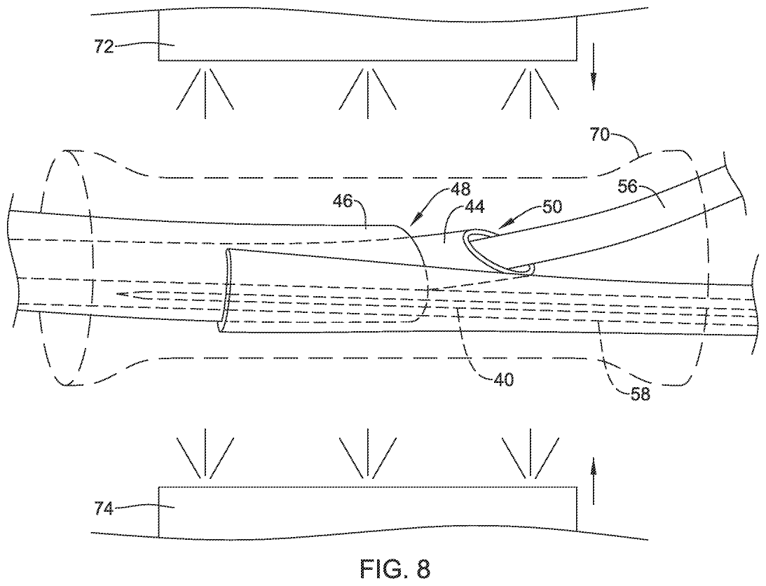

FIG. 7 shows a heat shrink tube 70 disposed over the assembly 68. FIG. 7A is a cross-sectional view taken along the line 7A-7A of FIG. 7. The heat shrink tube 70 is shown schematically, and may have a tighter fit to the assembly 68 even before application of any heat and/or pressure. FIG. 8 shows the assembly 68, along with the heat shrink tube 70, disposed between heat blocks 72 and 74. The heat blocks 72 and 74 emit heat in order to reduce the diameter of the heat shrink tube 70 to tightly fit around the outer perimeter of the assembly 68. The heat shrink 70 may have a length sufficient to span the entire region which will be subject to the reflow process that joins the proximal end region of the distal inner tubular shaft 44, the proximal end region of the distal outer tubular shaft 46 and the distal end region of the proximal tubular shaft 60 together in forming the port joint. In some cases, the heat blocks 72 and 74 move towards the assembly 68 and the heat shrink tube 70 in order to provide pressure on the assembly 68 and the heat shrink tube 70.

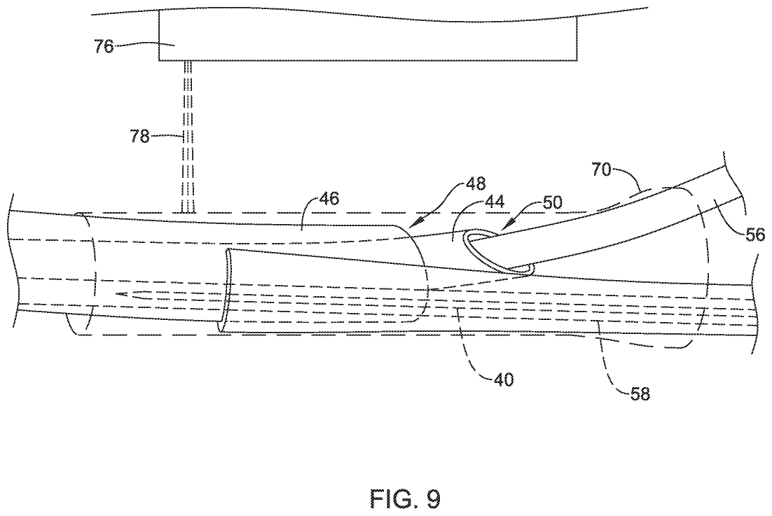

Once the heat shrink tube 70 has been secured around the assembly 68, heat may be applied to the region to melt the polymer materials of the proximal end region of the distal inner tubular shaft 44, the proximal end region of the distal outer tubular shaft 46 and the distal end region of the proximal tubular shaft 60 to allow the polymer materials to melt and reflow together. In some instances, a laser may be used to provide the heat during the reflow process. As shown in FIG. 9, a laser 76 provides a laser beam 78 that provides additional heating to the heat shrink tube 70 and the assembly 68. As a result, the heat shrink tube 70 undergoes a further reduction in diameter, thereby applying additional compressive force to the assembly 68. The combination of heat and pressure cause reflow to occur. As a result, portions of the proximal end region of the distal inner shaft 44, the proximal end region of the distal outer shaft 46, the flared distal end region 62 of the proximal shaft 60, including the tab 66 at least partially melt and reflow together, thereby creating a port joint in the catheter body 42 at the proximal guidewire port 34 of the catheter shaft 12 as shown in FIG. 2, with the distal end region of the core wire 40 encapsulated within the catheter body 42. The crescent-shaped lumen 38 extending through the port joint may be in fluid communication with the lumen of the proximal shaft 60 and the annular lumen between the inner surface of the distal outer shaft 46 and the outer surface of the distal inner shaft 44 distal of the port joint. The length of the port joint in which the proximal end region of the distal inner tubular shaft 44, the proximal end region of the distal outer tubular shaft 46 and the distal end region of the proximal tubular shaft 60 are reflowed together may be about 0.5 millimeters (mm) to about 25 mm, in some instances. As a consequence of the reflow process, the distal end region of the core wire 40 may be secured within the catheter body 42 of the catheter shaft 12 distal of the proximal guidewire port 34 without the use of any adhesive or other component other than the proximal end region of the distal inner tubular shaft 44, the proximal end region of the distal outer tubular shaft 46 and the distal end region of the proximal tubular shaft 60. In some cases, a distal tip of the core wire 40 may extend distally a short distance from the secured distal end region of the core wire 40 within the inflation lumen defined between the inner surface of the distal outer tubular shaft 46 and the outer surface of the distal inner tubular shaft 44. For example, the secured region of the core wire 40, where the core wire 40 is captured by the reflowed polymer about the core wire 40, may have a length of about 0.5 mm to about 25 mm, about 1 mm to about 10 mm, about 2 mm to about 5 mm, about 2 mm to about 4 mm, or about 2.5 mm, and the length of the core wire 40 extending distally of the secured region may have a length of about 0.5 centimeters (cm) to 50 cm, about 3 cm to about 7 cm, about 4 cm to about 6 cm, about 5 cm to about 6 cm, or about 5.5 cm, in some instances. In some instances, the core wire 40 may not extend distally of the secured region.

The devices described herein, as well as various components thereof, may be manufactured according to essentially any suitable manufacturing technique including molding, casting, mechanical working, and the like, or any other suitable technique. Furthermore, the various structures may include materials commonly associated with medical devices such as metals, metal alloys, polymers, metal-polymer composites, ceramics, combinations thereof, and the like, or any other suitable material. These materials may include transparent or translucent materials to aid in visualization during the procedure. Some examples of suitable metals and metal alloys include stainless steel, such as 304V, 304 L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A rapid exchange catheter, comprising:
an elongate shaft extending from a proximal end to a distal end;
a catheter hub disposed at the proximal end of the elongate shaft;
a guidewire port formed within the elongate shaft at a position distal of the proximal end of the elongate shaft and proximal of the distal end of the elongate shaft; and
a core wire extending past the guidewire port from a position proximal of the guidewire port to a position distal of the guidewire port;
the core wire including a distal end region secured to the elongate shaft distal of the guidewire port.

2. The rapid exchange catheter of claim 1, wherein the distal end region of the core wire is secured to the elongate shaft distal of the guidewire port without using any adhesive.

3. The rapid exchange catheter of claim 1, wherein the distal end region of the core wire is secured to the elongate shaft distal of the guidewire port by virtue of one or more components of the elongate shaft being reflowed around the core wire during manufacture of the rapid exchange catheter.

4. The rapid exchange catheter of claim 3, wherein the elongate shaft comprises a distal inner shaft having an outer diameter (OD), a distal outer shaft having an inner diameter (ID) and a proximal shaft positioned prior to reflowing with:
the distal inner shaft disposed within the distal outer shaft and extending proximally from the distal outer shaft;
the proximal shaft surrounding a proximal end of the distal outer shaft and adjacent to a proximal end of the distal inner shaft; and
the core wire disposed between the OD of the distal inner shaft and the ID of the distal outer shaft.

5. The rapid exchange catheter of claim 3, wherein prior to reflowing:
a port mandrel is inserted through the distal inner shaft; and
a crescent-shaped mandrel is inserted between the OD of the distal inner shaft and the ID of the distal outer shaft.

6. The rapid exchange catheter of claim 1, wherein the elongate shaft includes a distal outer shaft having a lumen, a distal inner shaft extending through the lumen of the distal outer shaft, and a proximal shaft extending proximal of the distal outer shaft and the distal inner shaft, wherein the core wire extends through a lumen of the proximal shaft such that the distal end region of the core wire is secured directly between an outer surface of the distal inner shaft and an inner surface of the distal outer shaft.

7. The rapid exchange catheter of claim 6, wherein the distal end region of the core wire is embedded between the distal inner shaft and the distal outer shaft.

8. The rapid exchange catheter of claim 1, wherein the elongate shaft includes a crescent-shaped lumen extending both proximal and distal of the guidewire port.

9. The rapid exchange catheter of claim 8, wherein a centroid of the distal end region of the core wire is laterally offset from a longitudinally extending plane passing through both a centroid of the elongate shaft and a centroid of the crescent-shaped lumen.

10. The rapid exchange catheter of claim 9, wherein the crescent-shaped lumen defines a portion of an inflation lumen extending through the elongate shaft to a balloon secured to a distal end of the elongate shaft.

11. A rapid exchange catheter, comprising:
an elongate shaft extending from a proximal end to a distal end;
a catheter hub disposed at the proximal end of the elongate shaft;
a port joint formed along the elongate shaft between the proximal end of the elongate shaft and the distal end of the elongate shaft;
the elongate shaft including a proximal shaft extending proximal of the port joint, a distal inner shaft extending distal of the port joint, and a distal outer shaft extending distal of the port joint, wherein a distal end of the proximal shaft is secured to a proximal end of the distal inner shaft and a proximal end of the distal outer shaft at the port joint;
wherein a lumen of the distal inner shaft opens to an exterior of the elongate shaft at a guidewire port formed at the port joint; and
a core wire extending distally past the guidewire port, the core wire including a distal end region secured to the elongate shaft at the port joint.

12. The rapid exchange catheter of claim 11, wherein the distal end region of the core wire is secured to the elongate shaft at the port joint without using any adhesive.

13. The rapid exchange catheter of claim 11, wherein the distal end region of the core wire is secured to the elongate shaft at the port joint by virtue of a proximal end region of the inner distal shaft and a proximal end region of the outer distal shaft being reflowed around the core wire during manufacture of the port joint.

14. The rapid exchange catheter of claim 11, wherein in a cross-section taken through the port joint perpendicular to a central longitudinal axis of the elongate shaft, the cross-section includes a guidewire lumen having a centroid spaced away from the central longitudinal axis of the elongate shaft in a first direction from the central longitudinal axis of the elongate shaft and a crescent-shaped inflation lumen having a centroid spaced away from the central longitudinal axis of the elongate shaft in a second direction from the central longitudinal axis of the elongate shaft, the second direction being opposite the first direction.

15. The rapid exchange catheter of claim 14, wherein in the cross-section the core wire has a centroid spaced away from the central longitudinal axis of the elongate shaft in a third direction from the central longitudinal axis of the elongate shaft, the third direction being different from the first direction and the second direction.

16. The rapid exchange catheter of claim 15, wherein the third direction is at an acute angle to the first direction.

17. A method of manufacturing a catheter having a rapid exchange guidewire port, the method comprising:
forming an assembly by:
disposing a distal inner shaft having an outer diameter (OD) within a distal outer shaft having an inner diameter (ID);
disposing a distal end region of a proximal shaft around a proximal end region of the distal outer shaft with a proximal end region of the distal inner shaft extending adjacent to the distal end region of the proximal shaft;
inserting a first mandrel through a lumen of the distal inner shaft;
inserting a second mandrel between the OD of the distal inner shaft and the ID of the distal outer shaft; and
inserting a core wire between the OD of the distal inner shaft and the ID of the distal outer shaft; and
thereafter, applying heat and pressure to the assembly in order to form the rapid exchange guidewire port.

18. The method of claim 17, wherein the distal end region of the proximal shaft includes an enlarged profile including a tab that extends along the OD of the distal inner shaft when the distal end region of the proximal shaft is disposed around the proximal end region of the distal outer shaft.

19. The method of claim 18, wherein the core wire extends proximally of the rapid exchange guidewire port.

20. The method of claim 17, wherein applying heat and pressure to the assembly causes the distal inner shaft, the distal outer shaft and the proximal shaft to melt and reflow around the first mandrel, the second mandrel and the core wire, thereby locking the core wire in place relative to the rapid exchange guidewire port.
